(19) United States
(12) Patent Application Publication
(10) Pub. No.: US 2019/0380647 A1
(43) Pub. Date: Dec. 19, 2019

(54) Biometric sensors and headsets

(71) Applicant: Artise Biomedical Co., Ltd., Zhubei City (TW)

(72) Inventors: Jung-Sheng Wang, Changhua County (TW); Shao-Wei Lu, Hsingchu County (TW); Cheng-Chieh Hung, Taipei City (TW); Jui-Huang Cheng, Hsinchu City (TW)

(73) Assignee: Artise Biomedical Co., Ltd., Zhubei City (TW)

(21) Appl. No.: 16/441,079

(22) Filed: Jun. 14, 2019

Related U.S. Application Data
(60) Provisional application No. 62/685,310, filed on Jun. 15, 2018.

(51) Int. Cl. A61B 5/00; A61B 5/0478

(52) U.S. Cl. CPC A61B 5/6803; A61B 5/6814; A61B 5/0478

(57) ABSTRACT

The problem of poor signal quality of biometric measurement is improved with a new headset construction. The new headset includes a sensor assembly that provides more stable and secure contact between the sensor electrodes and the scalp of the wearer of the headset. The sensor assembly includes a housing unit for the electrodes and has a flexible and collapsible wall portion, which flexes and deforms according to the strength and the location of the pressure applied to the electrodes to maximize the contact area between the scalp and the sensors.

### BACKGROUND

Traditional biomatrix measurements such as electroencephalogram (EEG) uses wet sensors in which conductive electrode plates are held on the human scalp by conductive gel. Recently, new measurement systems with dry sensors (without the aid of conductive gel) have gain limited acceptance, especially in the wearable EEG market for the added mobility of the wearer.

One drawback of the dry sensors is that the quality of the signal in terms of signal-to-noise ratio (SNR) is still inferior compared to wet sensors because without the aid of conductive gel, the dry sensors are more difficult to make and maintain good contact to the scalp. When signal quality and reliability are essential, uses still prefer systems with wet sensors.

Since good contact is necessary for good signal quality, traditional headset may require increased pressure between the sensor and the scalp but excessive pressure makes wearable EGG headsets uncomfortable to the wearer. This has hindered the acceptance of headsets with dry sensors.

### BRIEF SUMMARY OF THE INVENTION

The Inventors researched and discovered that the root cause of poor signal quality from traditional biometric measurement systems equipped with dry sensors, especially among the wearable headsets, is the difficulty in maintaining good conformity of the sensor surfaces, which have limited flexibility, to the scalp surface of human heads. The non-flatness of the scalp surface limits the surface area of the sensor that is active in collecting signals from the brain results in poor signal strength and the lack of consistency of signal quality.

Probes of biometric sensor usually come in two types—one type of probe is made of a conductive strip, such as a strip of copper, a second type is made of pin or a group of pins such as a plurality of conductive pogo pins fixed to a rigid base. Both types of sensors have limited intrinsic flexibility so that when the sensor is placed on the head of a wearer, the surfaces of the sensor electrodes and the scalp do not automatically align. Because the scalp surface is usually curved and the curvature varies from location to location and from one human head to the next. The existing sensors do not have a mechanism for keeping the electrodes at close to a normal (90°) angle to the scalp.

In order to remedy this problem, we endeavored to invent and herein disclose improved headsets for biometric measurement. The headsets comprise novel sensor assemblies to make and maintain good sensor electrode to scalp contact. The sensor assembly comprises a housing unit including a base portion, a foldable wall portion, and a top portion to which the electrodes are attached. The housing unit is unitary and is made of an elastic material. The collapsible wall portion is thin and thus can flex and collapse.

When the novel headset is initially worn by a wearer, due to the curvature of the scalp of the wearer, only a portion of the strip type electrode surface or only a portion of the pin type electrodes make good contact to the scalp. As the headset is being adjusted, the pressure exerted on the individual sensor assembly will be transferred to the collapsible wall portion of the housing unit and the wall will flex and collapse to effect a more perfect alignment between the electrode and the scalp surface. For example, if the initial placement of the electrode surface is not aligned to the scalp surface, the pressure exerted on the wall portion will be uneven. The unevenly asserted force on the collapsible wall portion will cause a portion of the wall to flex and collapse according to the force and thus change the contact angle between the electrode and the scalp to increase the contact area. When the exerted force exceeds a threshold, the entire wall will collapse and thus reduces the pressure and improves the comfort of the wearer.

### DEFINITION OF TERMS

The terms in this disclosure and claims have their ordinary meaning to a skill artisan. In order to describe the invention more clearly, the following terms are further defined.

BIOMATRIX is broadly defined as the theory of interacting biological systems. In this disclosure, a biometric system is more narrowly defined as a system for measuring biological activities, especially human brain activities.

HEADSET or BIOMETRIC HEADSET is a wearable piece of equipment designed to be worn by a human or other mammals. In this disclosure, it means a head gear fitted with biometric sensors and other communication components for the purpose of measuring the brain activities of the wearer.

ELECTRODE is a probe, usually electrically conductive, configured for sending to or collecting electrical signal from a non-conductive part of a system. In this disclosure, the electrodes are used to collect electric signals of brain activities of a subject.

ELECTRIC CIRCUIT BOARD or PRINTED CIRCUIT BOARD (PCB) is used to mechanically support and electrically connect electronic components or electrical components using conductive tracks, pads and other features etched from one or more sheet layers of copper laminated onto or between sheet layers of a non-conductive substrate. In this disclosure, electric circuit board connects the sensor elements on the headset to a transmitter or a transceiver on the headset to communicate with the brain of the subject.

SENSOR ASSEMBLY in this disclosure is defined as a combination of electrode or electrodes and a housing unit in which the electrodes are assembled.

### BRIEF DESCRIPTION OF THE DRAWINGS

Drawing figures in this disclosure depict exemplary embodiments of this invention, in which:

FIG. 1 depicts a biometric headset with sensor assemblies and supporting structure elements.

FIG. 2 depicts one portion of the headset depicted in FIG. 1.

FIG. 3 depicts the portion of headset of FIG. 2 with an explored view of one sensor assembly.

FIG. 4 depicts the operation of the sensor assembly depicted in FIG. 3

FIG. 5 depicts another senor assembly and associated supporting structure elements.

FIG. 6 depicts another portion of the headset depicted in FIG. 1.

FIG. 7 depicts another type of sensor assembly.

FIG. 8 depicts another type of sensor assembly.

FIG. 9 depicts another type of sensor assembly.

FIG. 10 depicts another type of sensor assembly.

FIG. 11 depicts another biometric headset with sensors and the supporting structure.

FIG. 12 depicts a wearer fitted with a biometric headset that embodies some aspects of this invention.

FIG. 13 depicts another wearer fitted with a biometric headset that embodies some aspects of this invention.

### DETAILED DESCRIPTION OF EXEMPLARY EMBODIMENTS

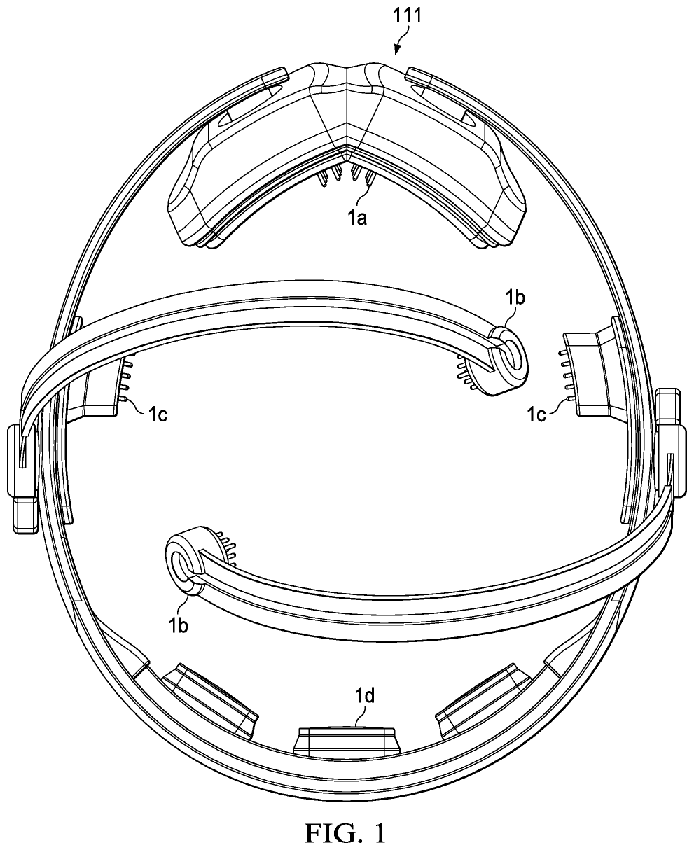

FIG. 1 depicts a biometric headset and sensor assemblies that embody some aspects of this invention. The headset includes four types of exemplary biometric sensor assemblies. 1a, 1b, 1c, and 1d. Each sensor assembly comprises a housing unit and an electrode or electrodes. The sensor assemblies are coupled to supporting structure members, which electrically connect the electrodes of the sensor assemblies to a PCB. The PCB and the electronic device components and the electric power source such as batteries can be housed in a housing unit 111 attached to one supporting structure member. The supporting structure members are physically connected to form the headset as depicted in FIG. 1.

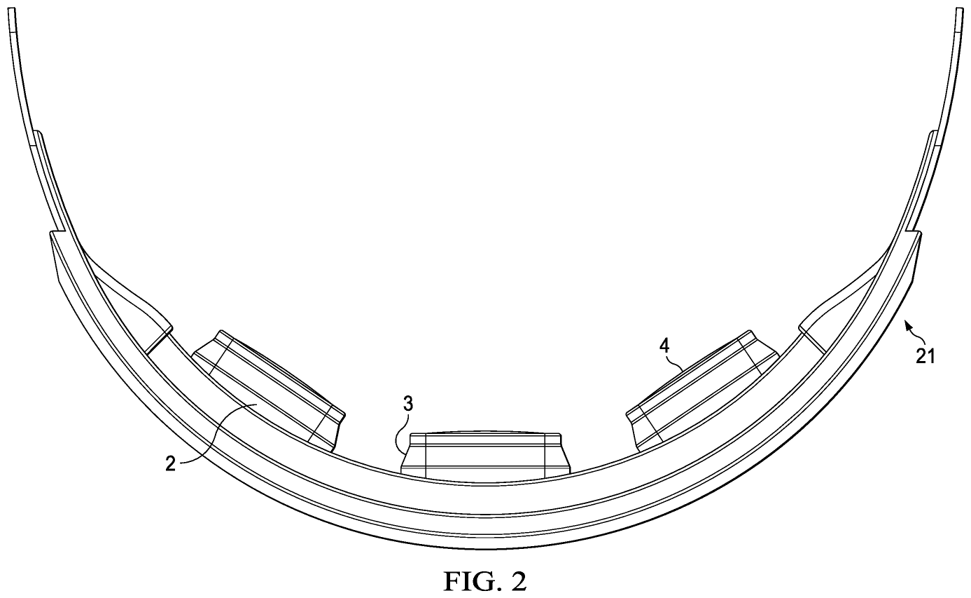

FIG. 2 depicts four sensor assemblies of type 1d attached on a supporting structure member 21. Each sensor assembly comprises a base portion 2, a collapsible wall portion 3, and a top portion 4. Type 1d sensor assembly has an oblong shape, and the supporting structure member is designed to fit to the forehead of a wearer.

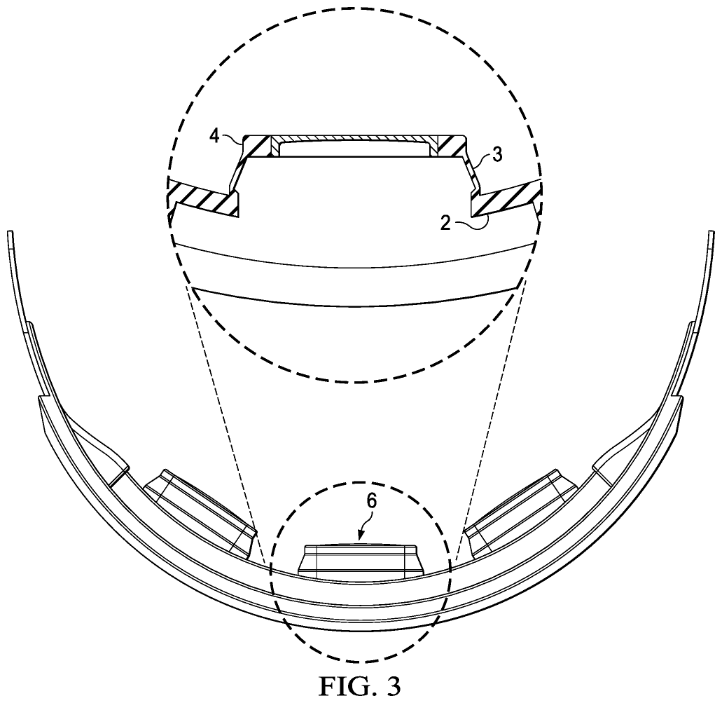

FIG. 3 further depicts the same type 1d sensor assembly as depicted in FIG. 2, with an exploded view of the housing unit. This exemplary house unit is constructed as a unitary unit of elastic material. The top member has a thickness 22 mm and is dimensioned to accommodate the electrode unit. The collapsible wall is about 1 mm thick and it flares out from the top member at with an obtuse angle of 113′2″ so it is flexible and is designed to collapse outwardly. In other embodiments, the wall may be constructed to flex and collapse inwardly with a different angle with respect to the base portion.

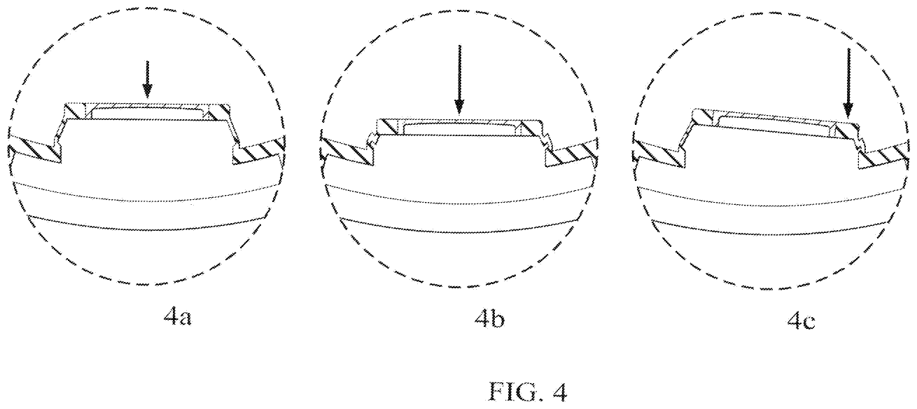

FIG. 4 depicts the operation of a collapsible wall portion 3 of the sensor assembly as depicted in FIG. 3. The drawing figure a on the left of FIG. 4 depicts a force being applied to the electrodes toward the base portion. If the applied force is less than a threshold value, the collapsible wall portion remains at its full height. The middle drawing b depicts the collapsible wall portion flexes and collapses when the applied force exceeds the threshold value. As the force is being applied to the center of the assembly, the wall flexes and collapses evenly so the surface of the electrodes stays aligned to the direction of the force being applied. The right drawing c depicts the force as being applied unevenly to the electrode—more to the right edge of the top portion—and the collapsible wall portion reacts by flexing and collapsing on the right edge.

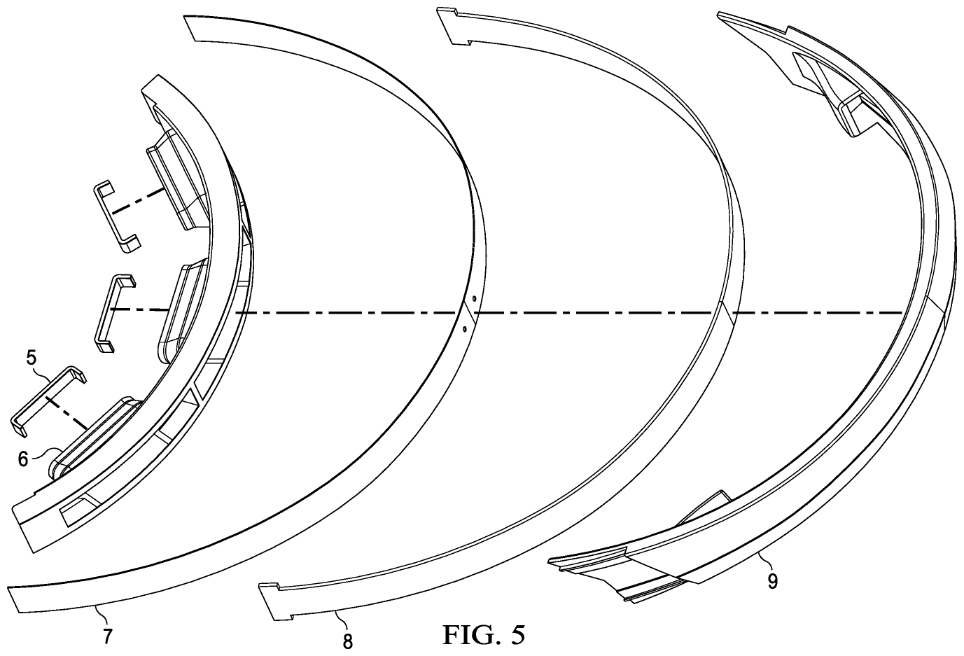

FIG. 5 depicts an exposed view of the sensor assembly type 1d and some of the associated supporting structure elements. In FIG. 5, the electrode 5 is a one-piece electrode housed in the housing unit 6. A PCB 7 is mechanically and electrically coupled to the electrodes 5. The PCB 7 is further mechanically supported by a flexible piece 8, which may be made of material such as steel. The PCB 7 and the supporting member 8 are enclosed in an outer piece 9, which may be made of plastic material for rigidity and limited flexibility.

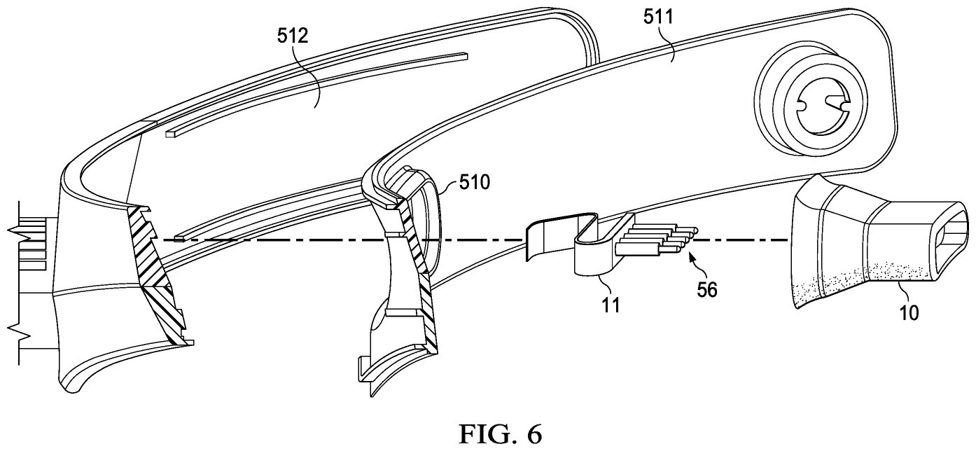

FIG. 6 depicts an exploded view of another sensor assembly type 1c and the associated supporting structure elements. The housing unit 10 comprises a top portion, a collapsible wall portion, and a base portion similar to that of the sensor assembly 1d described previously except the housing unit 10 has an oblong shape, similar to that in FIG. 5. The electrodes 56 of this sensor assembly are pogo pins, of which the total length may vary as pressure is applied to the tips. The electrodes 56 are held by and electrically coupled to a conductive piece 11, which fits an opening 510 in a frame members 511 and 512.

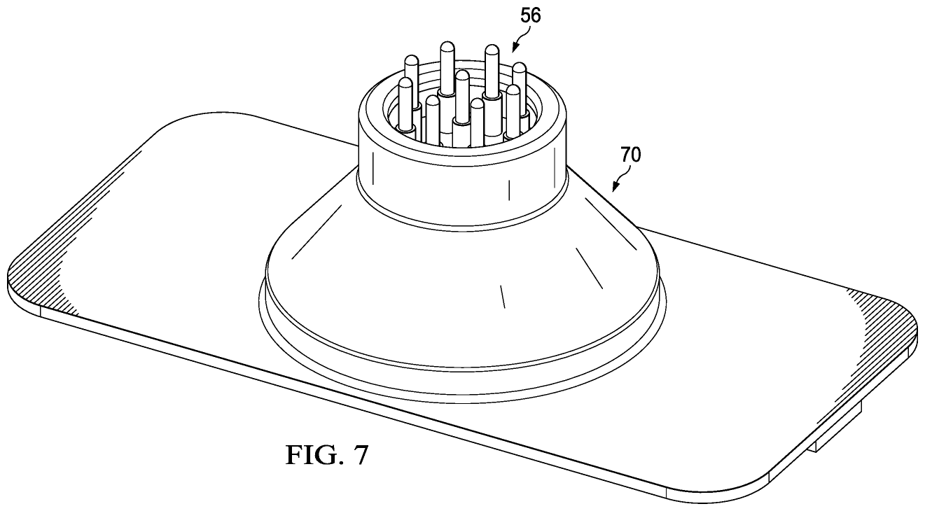

FIG. 7 depicts another sensor assembly type 1b, which is similar to the sensor assembly type 1c, except the round housing unit, which includes a base portion, a collapsible wall portion.

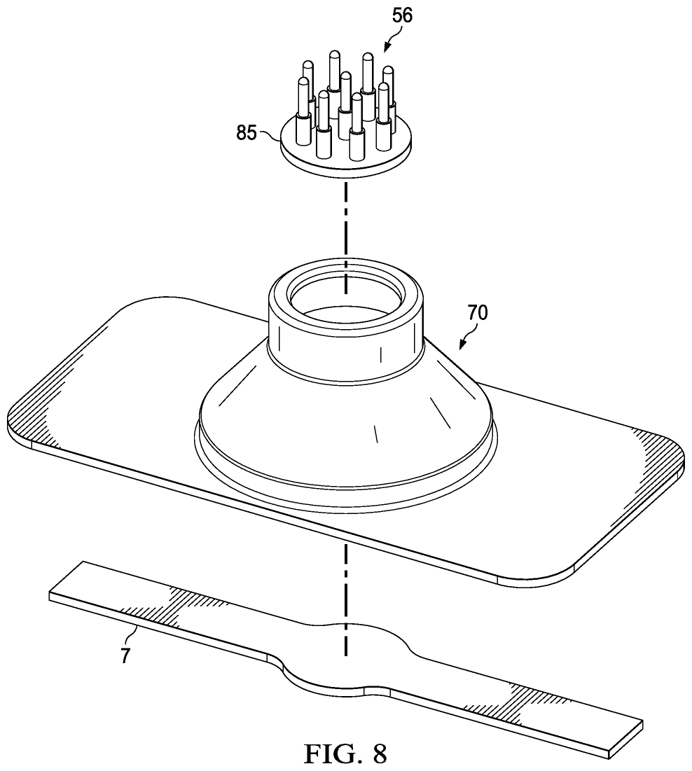

FIG. 8 depicts an exploded view of the sensor assembly type 1b, in which the electrodes 56 are embodied in a disk shape holder 85 that fits into the head portion of a round housing unit 70 of sensor assembly. Also depicted in FIG. 8 is a PCB 7, which is electrically couple to the electrodes.

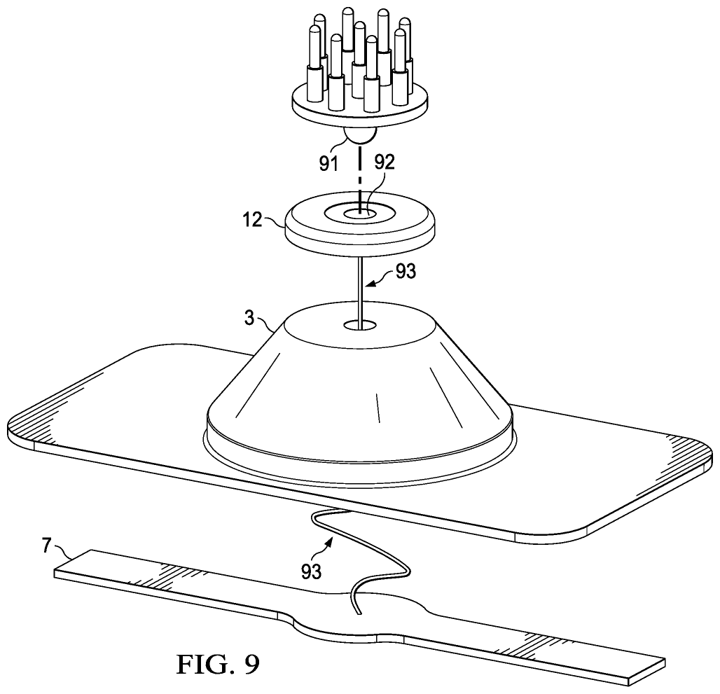

FIG. 9 depicts an exploded view of another version of the sensor assembly type 1b. The head portion 12 in this type has an opening 92 in the middle. A ball shape element 91 fits the opening and makes direct contact to a wire 93 that extends and connects to the PCB 7.

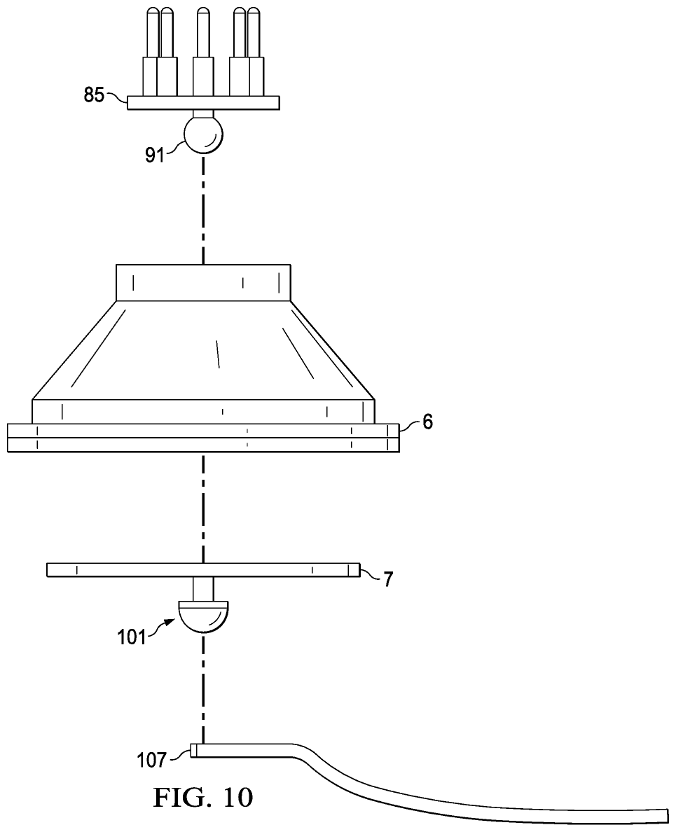

FIG. 10 depicts an exploded view of another version of the sensor assembly type 1c similar to the sensor assembly depicted in FIG. 9 except the PCB 7 only makes electrical contact to the ball shape member 91 when the wall portion of the housing unit 6 is collapsed and when the force applied on the electrodes exceeds a threshold value. The PCB 7 in turn is electrically coupled to a conductor 107.

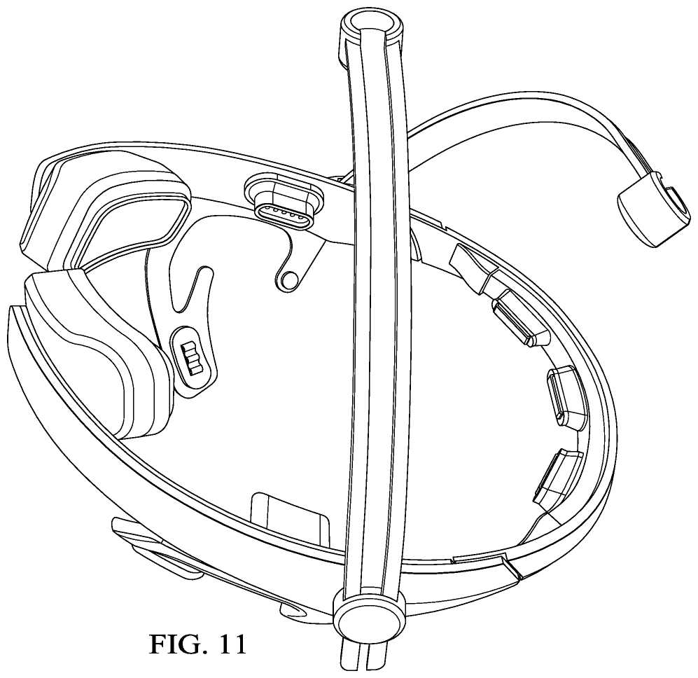

FIG. 11 another example of a headset that embodies some aspects of this invention depicts.

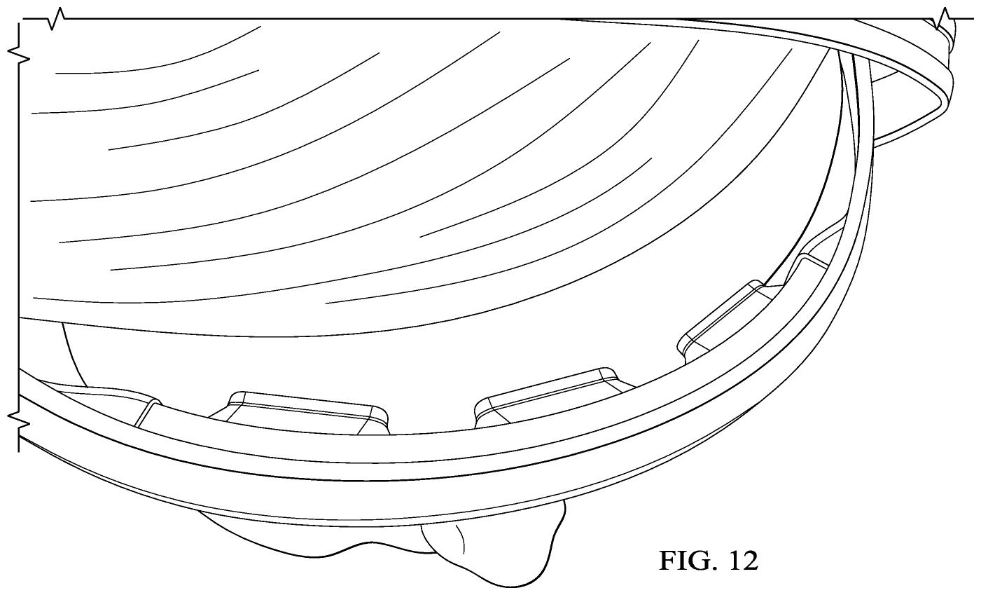

FIG. 12 depicts a wearer wearing a biometric headset that embodies some aspects of this invention.

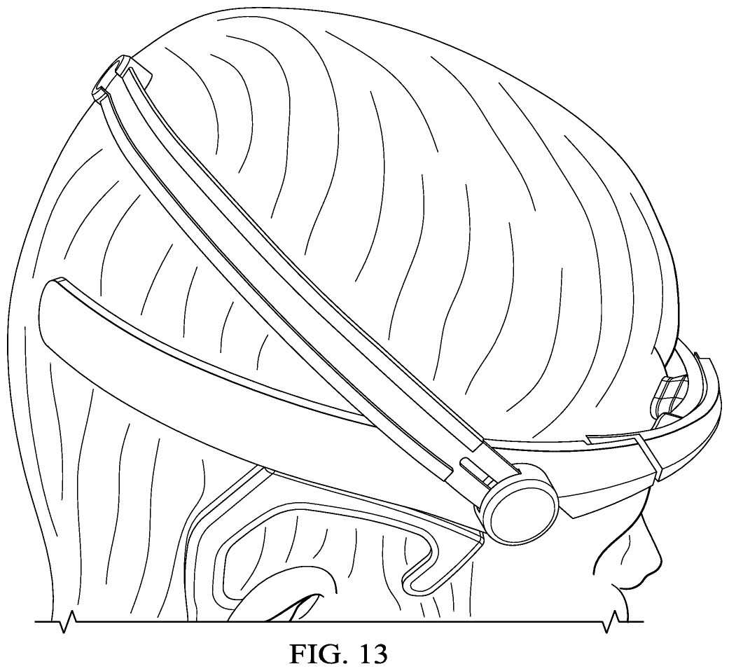

FIG. 13 depicts another wearer wearing another biometric headset that embodies some aspects of this invention.

We claim:

1. An headset comprising:
a plurality of biometric sensor assemblies:
each of the plurality of biometric sensor assemblies comprising one or more electrodes and a housing unit for housing the electrode or electrodes, the housing unit comprising a base portion, a top portion, and a collapsible wall portion between the base portion and the top portion;
the collapsible wall portion being thinner than the based portion and the top portion;
the electrode or electrodes configured to be held in the head portion; and
the collapsible wall portion configured to flex and to collapse towards the base portion when a force greater than a threshold force is applied at the electrode or electrodes.

2. The headset of claim 1, in which the collapsible wall portion is configured to flex and to collapse partially near the applied force that is applied unevenly at the electrode or electrodes.

3. The headset of claim 1, in which the base portion, the collapsible wall portion and the head portion are configured as a unitary unit.

4. The headset of claim 2, in which each electrode comprises a conductive metal strip.

5. The headset of claim 2, in which each electrode is configured as a pogo pin.

6. The headset of claim 5, in which each biometric sensor comprises a plurality of pogo pins.

7. The headset of claim 4, in which the collapsed wall portion collapses to maximize a surface area of the electrode in contact with a wearers scalp.

8. The headset of claim 6, in which the collapsed wall portion collapses to maximize the number of pogo pins in contact with a wearers scalp.

9. The headset of claim 1, in which the top portion has a round shape.

10. The headset of claim 1, in which the top portion has an oblong shape.

11. A biometric sensor assembly, comprising:
an electrode; and
a housing unit comprising
a base portion;
a top portion configured to hold the electrode; and
a collapsible wall portion between the base portion and the top portion, thinner than the based portion and the top portion.

12. The biometric sensor assembly of claim 11, in which the electrode comprising a conductive strip.

13. The biometric sensor assembly of claim 11, in which the electrode comprising a pogo pin.

14. The biometric sensor assembly of claim 13, in which the electrode comprising a plurality of pogo pins.

15. The biometric sensor assembly of claim 11, in which the top portion has a round shape.

16. The headset of claim 11, in which the top portion has an oblong shape.
